(19) United States
(12) Patent Application Publication
(10) Pub. No.: US 2006/0025840 A1
(43) Pub. Date: Feb. 2, 2006

(54) Cooling tissue inside the body

(76) Inventor: Martin Willard, Burnsville, MN (US)

Correspondence Address: FISH & RICHARDSON P.C., PO BOX 1022, MINNEAPOLIS, MN, 55440-1022, US

(21) Appl. No.: 10/909,695

(22) Filed: Aug. 2, 2004

(51) Int. Cl. A61F 7/12

(52) U.S. Cl. 607/113; 604/113

(57) ABSTRACT

Devices and methods for cooling a target tissue region inside the body are disclosed. A catheter for cooling blood includes an elongated member with a lumen extending longitudinally through a portion of the member. The lumen has an entry port and an exit port through which blood from a body vessel may enter and exit the lumen. An inflatable balloon is positioned between the entry and exit ports of the lumen, which when inflated, occludes the body vessel and prevent normal blood flow. A cooling element cools blood as it flows through the lumen. The cooling element may include a chamber that cools the blood by using a Joule-Thompson orifice to create a phase change of liquid to a gas. The cooling element may also include a thermoelectric cooler.

### TECHNICAL FIELD

This invention relates to cooling a target tissue region inside the body.

### BACKGROUND

Myocardial ischemia, and in severe cases acute myocardial infarction (AMI), can occur when there is inadequate blood circulation to the myocardium due to coronary artery disease. Evidence suggests that early reperfusion of blood into the heart, after removing a blockage to blood flow, dramatically reduces damage to the myocardium. However, the reestablishment of blood flow into the heart may cause a reperfusion injury to occur. Reperfusion injury is believed to be due to the build up of waste products on the myocardium during the time blood flow was inadequate and the reaction of these waste products with oxygen in the blood when normal blood flow is reestablished. It is possible to reduce reperfusion injury to the myocardium by cooling the myocardial tissue prior to reperfusion. Mild cooling of the myocardial tissue to a temperature between 28 and 36 degrees Celsius provides a protective effect, likely by the reduction in the rate of chemical reactions and the reduction of tissue activity and associated metabolic demands.

One method of cooling myocardial tissue is to place an ice pack over the patient's heart. Another method involves puncturing the pericardium and providing cooled fluid to a reservoir inserted into the pericardial space near the targeted myocardial tissue. Cooling of the myocardial tissue may also be accomplished by perfusing the target tissue with cooled solutions. A catheter having a heat transfer element located in the catheter's distal tip may also be inserted into a blood vessel to cool blood flowing into, and through, the heart. It is also possible to cool the myocardial tissue by supplying cool blood to the heart through a catheter placed in the patient's coronary sinus.

### SUMMARY

The invention features devices and methods to cool a target tissue region inside the body. In an aspect, the invention features a catheter that includes an elongated member with a lumen extending longitudinally through a portion of the member. The lumen has an entry port through which blood from a body vessel enters the lumen and an exit port through which the blood exits the lumen. An inflatable balloon is positioned between the entry and exit ports of the lumen, and when positioned within a body vessel and inflated, the balloon occludes the body vessel to prevent normal blood flow. A cooling element cools blood as it flows through the lumen.

In embodiments, the entry and exit ports of the lumen may be positioned so that when the catheter is in the body vessel, such as a coronary artery, the entry and exit ports are both within the body vessel. The inflated outer diameter of the inflatable balloon may be approximately five millimeters or less. The lumen may also be structured to provide a blood flow of twenty milliliters per minute through the lumen with normal blood pressure, and may also have a diameter of less than about 45 thousandths of an inch.

In other embodiments, the cooling element may be located in a distal portion of the catheter. The cooling element may include a chamber that cools the blood by using a Joule-Thompson orifice to create a phase change of liquid to a gas. The inflatable balloon can also include an inflation chamber, and the balloon's inflation chamber may also serve as the chamber that cools the blood using the Joule-Thompson orifice. In other embodiments, the cooling element includes a thermoelectric cooler, which may include a plurality of thermoelectric semiconductors.

In another aspect, the invention features a catheter for providing cooled blood to a target tissue region inside a body. The catheter includes an elongated member that has a lumen extending longitudinally through a portion of the member. The lumen has an entry port through which blood from a body vessel enters the lumen and an exit port through which blood exits the lumen. A chamber is positioned in a distal portion of the catheter between the entry and exit ports of the lumen so that the chamber may cool the blood as it flows through the lumen by using a Joule-Thompson orifice to create a phase change of liquid to a gas.

In embodiments, the entry and exit ports of the lumen may be positioned so that when the catheter is in the body vessel, such as a coronary artery, the entry and exit ports are both within the body vessel. In some embodiments, the chamber may also expand to occlude a body vessel to prevent normal blood flow to the target tissue region. The chamber may expand to an inflated outer diameter of approximately five millimeters or less.

In another aspect, the invention features a method of providing cooled blood to a target tissue region inside a body. A catheter that has an inflatable balloon near the catheter's distal end is introduced into a body vessel. The balloon is inflated to restrict normal blood flow to the target tissue region through the body vessel. Blood is allowed to flow through a lumen in the balloon catheter from an entry port proximal to the balloon to an exit port distal to the balloon, and the blood is cooled as it flows through the lumen.

In embodiments, the catheter may be positioned in the body vessel, for example a coronary artery, so that the entry and exit ports of the lumen are also within the body vessel. The method may also be performed during a percutaneous transluminal coronary angioplasty.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of a catheter in accordance with the invention.

FIG. 2 is a side cross-sectional view, in a longitudinal plane, of a distal portion of an embodiment of a catheter of the type shown in FIG. 1.

FIG. 3 is a cross-sectional view of the catheter along the line 3-3 shown in FIG. 2.

FIG. 4 is a perspective view of a thermoelectric cooler that may be used in a catheter in accordance with the invention.

FIG. 5 is a diagram of a side view of a distal portion of the FIG. 1 catheter positioned in a coronary artery, shown in cross-section, and illustrates a method of cooling a target tissue region in the heart.

FIG. 6 is a diagram of a side view of a proximal end of a catheter used to cool a target tissue region and a control system connected to the proximal end of the catheter, the control system shown in block diagram.

Like reference symbols in the various drawings indicate like elements.

### DETAILED DESCRIPTION

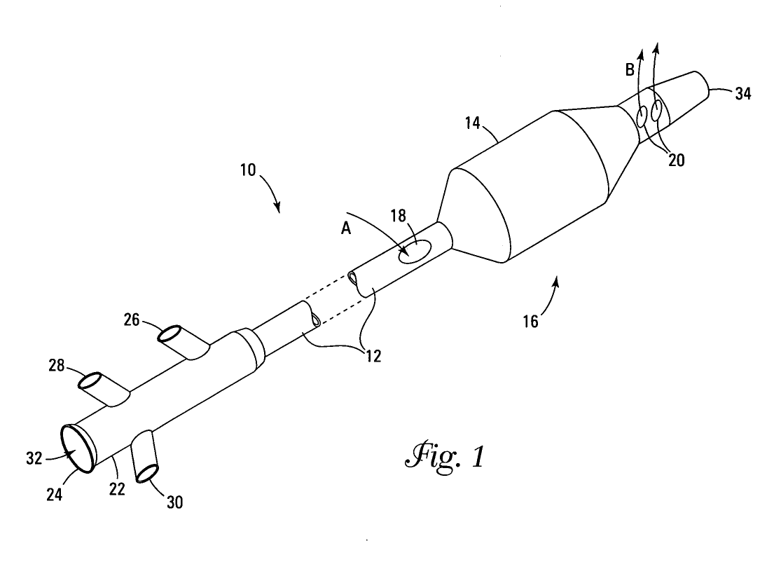

Referring to FIG. 1, a catheter 10 includes an elongate tubular shaft 12 and an inflatable balloon 14 at the catheter's distal portion 16. The catheter 10 may be used to repair a lesion in a body vessel, such as a coronary artery, that has reduced or completely blocked the flow of oxygenated blood to a tissue region. The catheter 10 may also be used to provide cooled blood to the oxygen-deprived, or ischemic, tissue region. A perfusion lumen (not shown in FIG. 1) extends longitudinally through the shaft 12 at the catheter's distal portion 16. When the balloon 14 is inflated in a body vessel so as to occlude blood flow, blood will be forced to enter the perfusion lumen through an entry port 18 in the catheter shaft 12 proximal to the balloon 14, as indicated by arrow A. A cooling element located in the catheter's distal portion 16 (not shown in FIG. 1) cools blood as it flows through the perfusion lumen, and the cooled blood exits the lumen distal to the balloon 14 through exit ports 20, as indicated by arrows B.

Delivery of cooled blood to the ischemic tissue region reduces the injury associated with the reperfusion of blood to the region without extending the time that the tissue region is deprived of oxygen. Because the blood provided to the tissue region during the cooling process is oxygenated, the cooling can be performed for as long as desired. Further, the oxygenated blood provided by the catheter 10 is cooled inside the body, and is not removed and cooled outside the body, which may damage blood cells. In addition, providing blood to the tissue region does not require the removal of the catheter's guide wire (not shown in FIG. 1) to infuse fluid into the vessel, which may compromise the position of the catheter 10 during a procedure.

An adapter 22 is attached to the shaft 12 at the catheter's proximal end 24. The adapter includes a longitudinal opening 32 at the proximal end 24, which provides access to a lumen (not shown in FIG. 1) inside the shaft 12. This internal lumen extends through the entire length of the shaft 12 to another longitudinal opening at the catheter's distal end 34. A guide wire (not shown) may be inserted through this internal lumen to allow a physician to maneuver the catheter through a body vessel and near a target tissue region. Once the catheter 10 is positioned, the guide wire may be removed and the lumen may also be used to provide fluid to the target tissue region.

The adapter 22 also includes ports 26, 28, and 30. The ports 26, 28, and 30 may provide access to lumens or wires connecting internal devices, such as a temperature sensor, that extend longitudinally through the catheter shaft 12 to the catheter's distal portion 16. The number of ports in the adapter, and the use of the ports, depends upon the type of cooling element used to cool the blood flowing through the perfusion lumen, as will be described in detail later.

In the FIG. 1 example, the catheter 10 may cool blood flowing through the perfusion lumen to a range of 25 to 36 degrees Celsius. The amount of cooling depends upon a number of factors, such as the volume flow rate of the blood through the perfusion lumen, the length and inside diameter of the perfusion lumen, and the cooling capability of the cooling element. For example, in an implementation where the length of the perfusion lumen is approximately 20 millimeters and the perfusion lumen's inside diameter is approximately 40 thousandths of an inch, the volume flow rate of blood through perfusion lumen is approximately 24 ml/min. Also in this example, the temperature of the cooling element is approximately minus 10 degrees Celsius, the blood flowing through the perfusion lumen can be cooled from normal body temperature of approximately 37 degrees Celsius to approximately 29 degrees Celsius. The cooling of the blood may be varied by changing one or more of these variables. For example, by reducing the volume flow rate of the blood through the perfusion lumen to 12 ml/min, with all other things remaining constant, the blood may be cooled to 25 degrees.

The volume flow rate of blood through the perfusion lumen is determined by the size of the perfusion lumen, the size and shape of the entry port 18 and the exit ports 20, and, of course, the blood pressure at the entry port 18. In the FIG. 1 implementation, the entry port 18 has a substantially oval shape and with axes of approximately 4.5 and 1.5 millimeters. In other implementations, the entry port 18 may be configured in another shape and the surface area of the port 18 may be increased or decreased. Further, additional entry ports may be added to the catheter 10 to allow additional blood flow to enter the perfusion lumen. The FIG. 1 catheter has two oval-shaped exit ports 20 with axes of approximately five hundredths and two hundredths of an inch. Like the entry port 18, the exit ports 20 may also be configured in another shape and the combined surface area of the exit ports 20 may be increased or decreased as desired. In addition, additional exit ports may be added to the catheter shaft 12, or alternatively, the shaft 12 may have only one exit port. In examples where the blood flow rate through the perfusion lumen is reduced to increase the cooling of the blood, inflation/deflation cycling of the balloon 14 may be required to oxygenate the tissue distal to the balloon. To prevent reperfusion injury, however, the balloon 14 should not be deflated to allow oxygenated blood at body temperature to reach the tissue region until the tissue region has first been cooled.

The catheter 10 may cool blood flowing through the perfusion lumen with a variety of different cooling elements or mechanisms, depending upon factors such as the length of the perfusion lumen, the desired amount of cooling, the desired size of the catheter's distal portion 16, and the flexibility of the distal portion 16 of the catheter required for the specific application. The cooling element may be, for example, a chamber that is positioned adjacent to the perfusion lumen and is accessible via one or more lumens in the catheter. In this example, a cool fluid may be provided to the chamber, which in turn cools the blood flowing through the perfusion lumen.

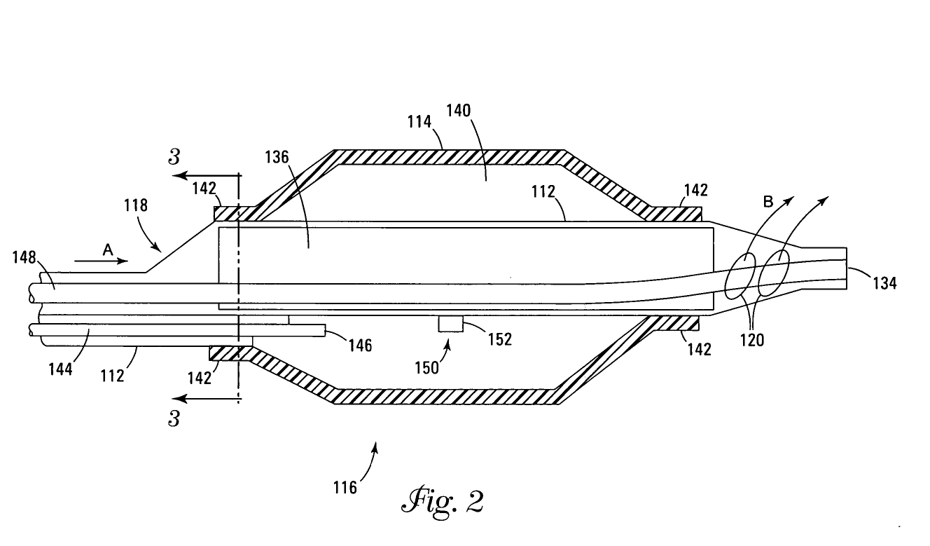

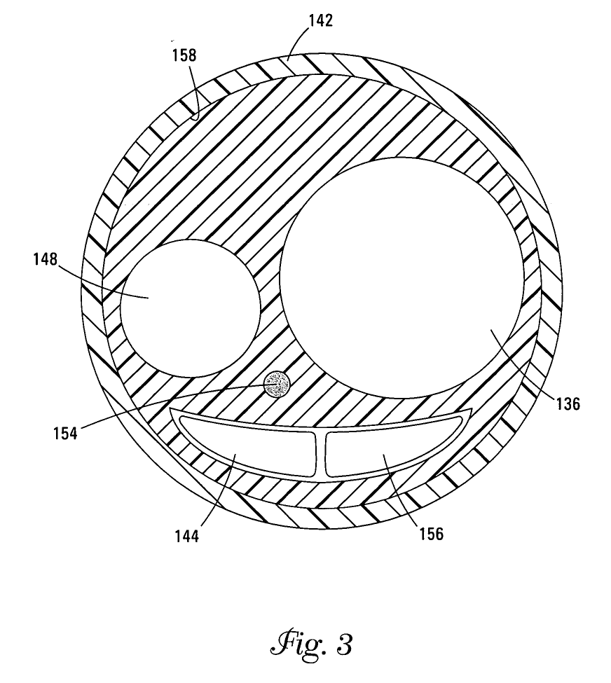

In another embodiment, a chamber may be used to cool the blood that flows through the perfusion lumen using a physical process called the Joule-Thompson effect. To use this process, a highly-pressurized fluid is introduced into the chamber and is allowed to change phase from a liquid to a gas across an orifice located at a distal end of a lumen. As the fluid changes phase, energy in the form of heat is pulled form the surrounding area, which cools the chamber and the blood flowing through the perfusion lumen. An example of a catheter that uses the Joule-Thompson effect to cool blood is shown in FIGS. 2 and 3.

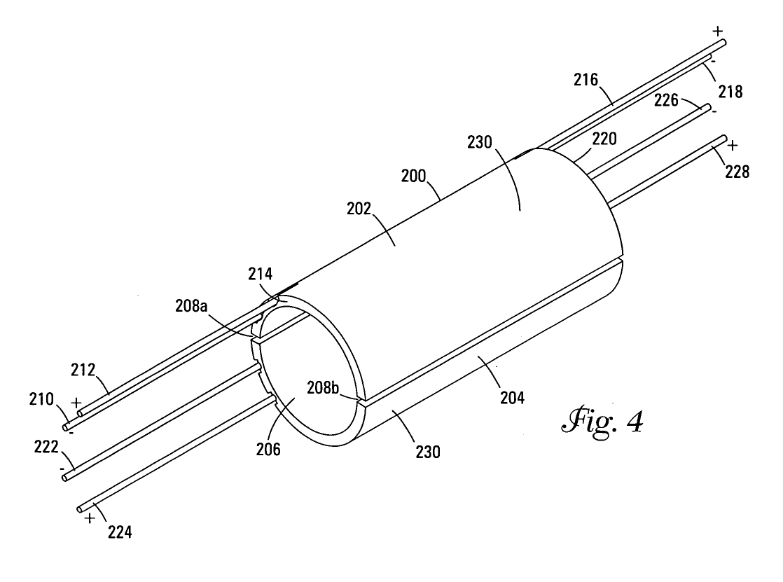

In other implementations, the cooling element may be thermoelectric cooler (TEC) (shown in FIG. 4), which cools blood flowing through the perfusion lumen using a process called the Peltier effect. In this example, the TECs are positioned between the entry port 18 and exit ports 20 and in thermal contact with the blood flowing through the perfusion lumen, as will be discussed later. The TECs that are currently available do not have the cooling capability of a Joule-Thompson cooling element of a similar size and cooling surface area. As a result, current TECs may not be capable of cooling blood to 29 degrees Celsius as in the previous example where the length of the perfusion lumen was 20 millimeters with an inside diameter of 40 thousandths of an inch and the volume flow rate of the blood through perfusion lumen is 24 mmin. Thus, to achieve the same amount of cooling, TECs may currently be used only in applications where the volume flow rate of blood is reduced or the length of the perfusion lumen is increased. As the cooling ability of TECs continues to increase, they may become suitable for more applications in the future.

FIG. 2 is a side cross-sectional view, in a longitudinal plane, of a distal portion 116 of a catheter that uses the Joule-Thompson effect to cool blood as it flows through the catheter's perfusion lumen 136. The catheter's distal portion 116 includes an inflatable balloon 114 that is positioned over a shaft 112 between the entry port 118 and the exit ports 120 of the perfusion lumen 136, and around the shaft's entire circumference. Welds (not shown) secure and seal the longitudinal ends 142 of the balloon 114 to the shaft 112, thus forming a sealed chamber 140 between the shaft 112 and the balloon 114. An infusion lumen 144 extends through the shaft 112, from a port in an adapter (e.g., the port 26 of FIG. 1) to, and into, the sealed chamber 140. A highly pressurized fluid, such as CO₂, N₂O, N₂, or He, is introduced into the sealed chamber 140 and expands into a gas across a Joule-Thompson orifice 146.

The phase change performs two functions in the FIG. 2 catheter. In addition to reducing the temperature of the chamber 140, the phase change to gas also inflates the balloon 114, which may repair a lesion in a body vessel, if necessary, and also block normal blood flow through the body vessel and force the blood into the perfusion lumen 136. An exhaust lumen (shown in FIG. 3), which extends longitudinally from the sealed chamber 140 to an adapter port (e.g., the port 28 shown in FIG. 1), removes excess gas from the sealed chamber 140 to maintain a desired pressure in the chamber 140 and inflate the balloon 114 to a desired level.

In the FIG. 2 example, a temperature sensor 150 is located inside the chamber 140 and monitors the temperature of the chamber 140. In this example, the temperature sensor 150 is a thermocouple. The thermocouple consists of two conductive wires 154 of dissimilar material that are insulated from each other. The wires 154 extend longitudinally through the catheter shaft 112 from a port in an adapter, for example the port 30 in the adapter 22 shown in FIG. 1, and into the chamber 140. The conductive wires 154 are joined together to form a junction 152, which is in thermal contact with the gas inside the chamber 140. When two dissimilar conductors are joined in this manner, an electromotive force (emf) is induced across the junction 152, the magnitude of which varies as a function of the junction's temperature. The induced emf may be measured at the proximal ends of the conductive wires 154, and thus allow the temperature of the chamber 140 to be measured. In other implementations, the temperature sensor 150 may be a thermistor or other suitable temperature-sensing mechanism. The temperature sensor 150 may also be placed in different locations in the shaft 112 to measure the temperature of the chamber. In other implementations, additional temperature sensors may be added to the catheter to measure, for example, the temperature of the blood exiting the exit ports 120.

A lumen 148 extends longitudinally through the catheter from an opening at the catheter's proximal end (e.g., the longitudinal opening 32 shown in FIG. 1) to an opening in the catheter's distal end 134. A guide wire (not shown) may be extended longitudinally through this lumen 148 to allow a physician to guide the catheter's distal portion 116 through a body vessel to a target tissue region. Once the catheter is positioned in the body, the lumen 148 may also be used to provide fluid to the target tissue region if desired. For example, cool blood or a blood substitute could be provided to the target tissue region. Cool saline or a saline solution containing antioxidants or other vascular agents such as nitric oxide, lidocaine, nitroglycerine, insulin, etc., may also be provided via lumen 148.

In the FIG. 2 example, the walls of the balloon 114 have a greater thickness, for example 0.0015 inch, than typical inflation balloons for balloon catheters, which are approximately 0.0007 inch. The increased thickness of the balloon walls insulates bodily fluids and tissues that contact the outer surface of the balloon 114. The insulation may limit the systematic cooling effects of the catheter and improve the efficiency of the targeted cooling of the blood flowing through the perfusion lumen 136. In other implementations, the balloon thickness may be increased or decreased as required. Alternatively, an additional outer layer may be added to the balloon 114. The additional outer layer may be constructed of a polymer, for example, polyester. In some implementations, a fluid or a polymer material may be placed between the balloon 114 and the additional outer layer to provide an additional insulation.

FIG. 3 shows a cross-sectional view of the catheter's distal portion 116 at line 3-3 of FIG. 2 looking proximally from the balloon 114. The FIG. 3 cross-section illustrates the relative size and location of the perfusion lumen 136, the lumen 148 for the guide wire and infusion of fluid to the target tissue region, the infusion lumen 144 and exhaust lumen 156, and the conductive wires 154. The balloon's longitudinal end portion 142 is shown attached to the shaft's outer surface 158.

The perfusion lumen 136 may have a diameter of approximately 39 to 42 thousandths of an inch, and may vary depending upon the application. The diameter of the perfusion lumen 136 may be increased to increase the flow rate of blood through the lumen, or alternatively, the diameter may be decreased to reduce the flow rate of blood. The lumen 148 may have a diameter of approximately 15 to 20 thousandths of an inch, and may be increased or decreased depending upon the application and the type of guide wire a physician may want to use to perform the procedure.

The infusion lumen 144 and exhaust lumen 156 in the FIG. 3 example collectively form a half-circle in cross-section, with the infusion lumen 144 and exhaust lumen 156 each making up approximately half of the area. In other implementations, the infusion lumen 144 and exhaust lumen 156 may have circular cross-sections, or be constructed in another suitable configuration.

FIG. 4 is a perspective view of a TEC 200 that may be used to cool blood as it flows through a perfusion lumen for delivery to a target tissue region using a thermal energy process known as the Peltier effect. The TEC 200 includes a first and second module 202 and 204, which when placed together, form a cylinder with a lumen 206 through which blood may flow. The TEC 200 may be placed in the outer wall of the perfusion lumen so that the blood flows through the lumen 206 of the TEC 200 for cooling as it flows through the perfusion lumen.

To form this cylinder-shaped structure, both the first and second modules 202 and 204 are in the shape of a half-cylinder, where the cylinder is split longitudinally in two equally-sized sections. The longitudinal edges of the first and second modules 202 and 204 are separated by small gaps 208a and 208b.

The first module 202 of the TEC 200 is connected to wires 210 and 212 at the first module's proximal end 214, and connected to wires 216 and 218 at the first module's distal end 220. In this implementation, the wires extend 210 and 212 extend longitudinally through the shaft of the catheter toward the catheter's proximal end so that the temperature of the TECs may be controlled, as explained later. If the catheter includes additional TECs 200, then the wires 210 and 212 may be connected to the first module of another TEC. If the TEC 200 is the most proximal TEC in the catheter shaft, the wires 210 and 212 extend longitudinally through the shaft to the catheter's proximal end for access outside of the patient through a port in an adapter, for example the port 30 shown in FIG. 1. The wires 216 and 218 extend longitudinally through the catheter shaft toward the catheter's distal end and may be connected to the first module of another TEC located distal to the TEC 200.

The second module 204 is similarly connected to wires 222 and 224 at the second modules proximal end 214, and connected to wires 226 and 228 at the second module's distal end 220. The wires 222, 224, 226, and 228 extend longitudinally through the shaft and connect to the second modules of the various TECs in the catheter in the same manner as described for the first module 202.

The first and second modules 202 and 204 may, for example, contain a series of thermoelectric cooling elements. The elements may be, for example, packaged within an electrical insulator and include an n-type semiconductor and a p-type semiconductor connected in series. In other implementations, the semiconductors may be replaced with other suitable materials. The semiconductors would typically be arranged between a ceramic substrate that electrically insulates the conductors from heat sinks attached to the ceramic substrate on two sides of the thermoelectric cooling element. The thermo electric cooling elements are arranged so that one heat sink is adjacent to contact the internal surface of the modules 202 and 204 (i.e., the surface that forms the lumen 206). The other heat sink is arranged to be adjacent to the external surface 230 of the modules 202 and 204.

To utilize the cooling effect of the TEC 200, a DC voltage may be applied to the elements via the wires 210, 212, 222, and 224, which causes a current to pass through the semiconductor pairs. The current causes heat to be drawn from the heat sink on the surface that forms the lumen 206 to the heat sink near the external surface of the modules 230. Through this process, the internal surface that forms the lumen 206 is cooled, and at the same time, the external surface 230 is heated. By cooling the internal surface that forms the lumen 206, the blood flowing through the perfusion lumen of the catheter may also be cooled.

In an implementation where a TEC 200 is used for cooling, using both the infusion and exhaust lumens shown in FIGS. 2 and 3 may be unnecessary. A single lumen may be sufficient to inflate and deflate the balloon at the catheter's distal end. Like the FIG. 2 infusion lumen, the balloon inflation lumen may extend longitudinally from the sealed chamber formed by the balloon to a port in the catheter's adapter.

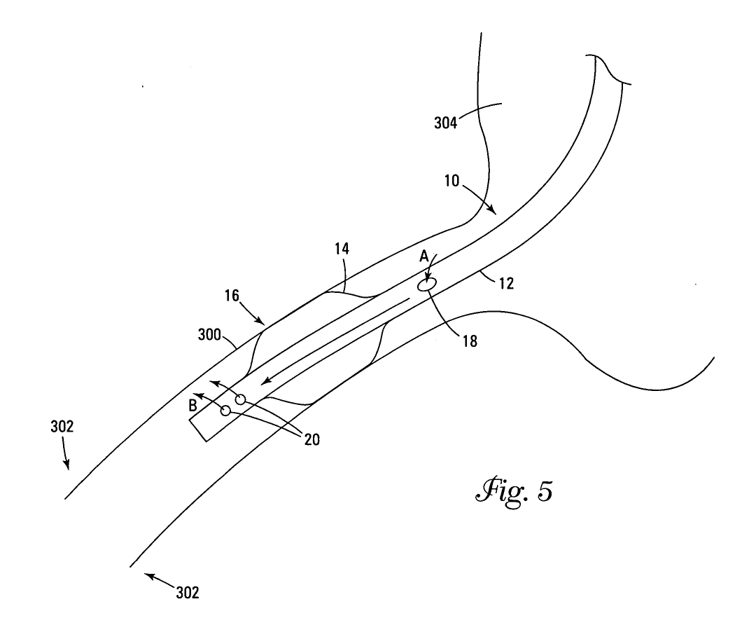

FIG. 5 is a diagram of a side view of a distal portion 16 of the FIG. 1 perfusion catheter positioned in a coronary artery, shown in cross-section, and illustrates a method of cooling a target tissue region 302 in the heart. In the FIG. 5 example, the distal portion 16 of the perfusion catheter 10 is positioned in a coronary artery 300 of the heart, via the aorta 304, that contains a lesion or blockage and is being treated with percutaneous transluminal coronary angioplasty. Once the distal portion 16 of the catheter is positioned in the artery 300, the balloon 14 is inflated to prevent normal blood flow to the target tissue region 302, and in some implementations, to open an occlusion of the artery 300. Blood that enters the perfusion lumen through entry port 18, as indicated by arrow A, is cooled by the cooling element in the catheter's distal portion 16. The blood then exits the perfusion lumen through exit ports 20, as indicated by arrows B, and is provided to the tissue region 302 to reduce reperfusion injury.

The FIG. 1 catheter may also be used to cool tissue regions in other areas of the body. For example, the catheter may be used in the brain, kidneys, and legs.

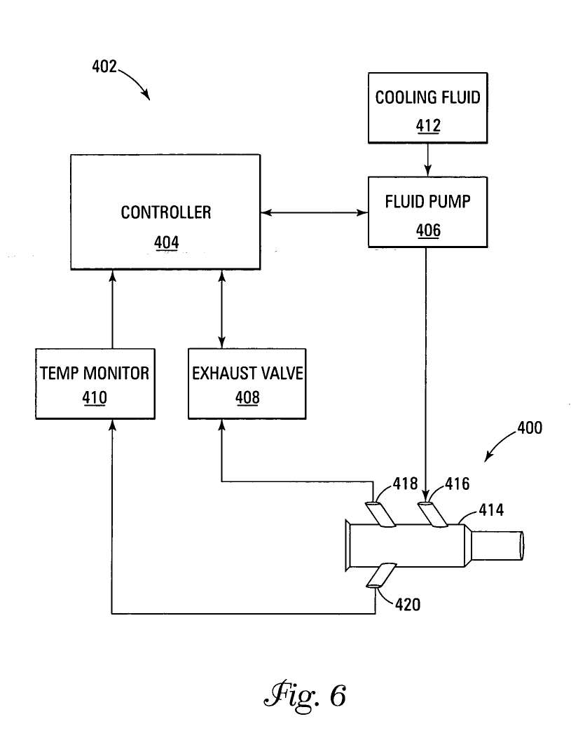

FIG. 6 shows a system including the previously described catheter (only a portion of which is shown in FIG. 6) and various external equipment attached to the catheter. In this example, the catheter is attached to a control system 402, which includes a controller 404, a fluid pump 406, an exhaust valve 408, and a temperature monitor 410. The controller 404 receives information from the temperature monitor 410 and uses that information to control the operation of the fluid pump 406 and the temperature of the blood delivered to a target tissue region. The controller 404 also monitors the pressure in the catheter's balloon (not shown in FIG. 6), which dictates the balloon's inflation and deflation, and also permits the continual expansion of gas into the balloon's chamber for cooling.

The catheter's proximal end 400 has an adapter 414 with ports 416, 418, and 420. The port 416 provides access to an infusion lumen that extends longitudinally through the catheter to the balloon's chamber in the catheter's distal portion. The fluid pump 406 is connected to the infusion lumen via port 416. The controller 404 controls the operation of the fluid pump 406, and thus the amount and rate of super-cooled fluid provided to the balloon's chamber. The super-cooled fluid 412 provided to the sealed chamber may be CO₂, N₂O, N₂, He, or another suitable fluid.

The port 418 provides access to an exhaust lumen that extends longitudinally through the catheter from the balloon's chamber. The exhaust valve 408 is connected to the exhaust lumen via port 418. The controller 404 controls and monitors the removal of gas from the balloon's chamber by exhaust valve 408. The port 420 provides access to a temperature sensor that senses the temperature of the sealed chamber. For example, in an implementation where the temperature sensor is a thermocouple (as shown in FIG. 2), the port 420 provides access to the conductive wires that extend from the thermocouple's junction in the distal portion of the catheter.

In other implementations, additional external devices may be added to the control system 402, or alternatively, some of the devices may be omitted. Further, the control system 402 may be modified to control the cooling of catheters that use a TEC to cool the blood flowing through the perfusion lumen. In such an implementation, the fluid pump 406 may be used to introduce and remove an inflation medium, and thus inflate and deflate the catheter's balloon. The exhaust valve may be replaced with a DC voltage source that controls the amount of cooling of the TECs. The temp monitor may be used to monitor a temperature sensor that measures the temperature of the fluid exiting the catheter's perfusion lumen.

A number of embodiments of the invention have been described. Nevertheless, it will be understood that various modifications may be made without departing from the spirit and scope of the invention. Accordingly, other embodiments are within the scope of the following claims.

What is claimed is:

1. A catheter comprising:
an elongated member that has a lumen extending longitudinally through a portion of the member, the lumen having an entry port through which blood from a body vessel enters the lumen and an exit port through which the blood exits the lumen;
an inflatable balloon positioned between the entry and exit ports of the lumen; and
a cooling element that cools blood as it flows through the lumen.

2. The catheter of claim 1 wherein the entry and exit ports of the lumen are positioned such that when the catheter is in the body vessel, the entry and exit ports are both within the body vessel.

3. The catheter of claim 2 wherein the body vessel is a coronary artery.

4. The catheter of claim 1 wherein the inflatable balloon, when inflated, has an outer diameter that is approximately five millimeters or less.

5. The catheter of claim 1 wherein the lumen is structured to provide a blood flow of twenty milliliters per minute through the lumen with normal blood pressure.

6. The catheter of claim 5 wherein the lumen has a diameter of less than about 45 thousandths of an inch.

7. The catheter of claim 1 wherein the cooling element is located in a distal portion of the catheter.

8. The catheter of claim 1 wherein the cooling element further comprises a chamber that cools the blood by using a Joule-Thompson orifice to create a phase change of liquid to a gas.

9. The catheter of claim 8 wherein the inflatable balloon comprises an inflation chamber, and wherein the balloon's inflation chamber serves as the chamber that cools the blood using the Joule-Thompson orifice.

10. The catheter of claim 1 wherein the cooling element comprises a thermoelectric cooler.

11. The catheter of claim 10 wherein the thermoelectric cooler comprises a plurality of thermoelectric semiconductors.

12. A method of providing cooled blood to a target tissue region inside a body, the method comprising:
introducing a catheter into a body vessel, the catheter having an inflatable balloon near the catheter's distal end;
inflating the balloon to restrict normal blood flow to the target tissue region through the body vessel;
allowing blood to flow through a lumen in the balloon catheter from an entry port proximal to the balloon to an exit port distal to the balloon; and
cooling the blood as it flows through the lumen.

13. The method of claim 12 wherein the catheter is positioned in the body vessel such that the entry and exit ports of the lumen are also within the body vessel.

14. The method of claim 13 wherein the body vessel is a coronary artery.

15. The method of claim 12 wherein the method is performed during a percutaneous transluminal coronary angioplasty.

16. A catheter for providing cooled blood to a target tissue region inside a body, the catheter comprising:
an elongated member that has a lumen extending longitudinally through a portion of the member, the lumen having an entry port through which blood from a body vessel enters the lumen and an exit port through which blood exits the lumen; and
a chamber positioned in a distal portion of the catheter between the entry and exit ports of the lumen so that the chamber may cool the blood as it flows through the lumen by using a Joule-Thompson orifice to create a phase change of liquid to a gas.

17. The catheter of claim 16 wherein the entry and exit ports of the lumen are positioned such that when the catheter is in the body vessel, the entry and exit ports are both within the body vessel.

18. The catheter of claim 17 wherein the body vessel is a coronary artery.

19. The catheter of claim 16 wherein the chamber expands to occlude a body vessel to prevent normal blood flow to the target tissue region.

20. The catheter of claim 19 wherein the inflated outer diameter of the chamber is approximately five millimeters or less.
